(19) United States
(12) Patent Application Publication
(10) Pub. No.: US 2005/0090905 A1
(43) Pub. Date: Apr. 28, 2005

(54) Porous implant with a dried, lubricious when wet, in vivo absorbable coating

(76) Inventors: Michael E. Hawkins, Columbia City, IN (US); Steven J. Charlebois, Goshen, IN (US)

Correspondence Address: ZIMMER TECHNOLOGY - ROBERTS;INDIANO, VAUGHN & ROBERTS, 1 NORTH PENNSYLVANIA AVENUE #850, INDIANAPOLIS, IN, 46204, US

(21) Appl. No.: 10/693,391

(22) Filed: Oct. 23, 2003

(51) Int. Cl. A61F 2/28

(52) U.S. Cl. 623/23.51

(57) ABSTRACT

An apparatus including a medical implant is provided. The medical implant includes a porous outer portion, and the apparatus further includes a dried, lubricious when wet, in vivo absorbable coating covering the porous outer portion. A method for finishing a medical implant is also provided. The method includes forming a porous outer portion on the implant, covering the porous outer portion with a lubricious when wet, in vivo absorbable coating, and drying the coating.

### FIELD OF THE INVENTION

The present invention relates generally to the field of medicine, and, more particularly, to a porous implant with a dried, lubricious when wet, in vivo absorbable coating.

### BACKGROUND

Some medical implants are manufactured to have “porous” outer portions. The “pores” (i.e., uniformly or irregularly spaced holes, cavities, or voids formed by meshes, lattices, or the like) can allow bone ingrowth over time, which enhances fixation. Alternatively or additionally, such pores can allow ingress of bone cement to enhance fixation at implantation. A variety of manners for constructing medical implants having porous outer portions (i.e., “porous implants”) are known. The porous portions may be formed, for example, by perforating a suitable material, or by sintering, diffusion bonding, or welding metal beads or metal fibers to form an integral part of the implant.

In any event, the outer surfaces of some porous implants are laced with rough or jagged edges that may injure soft tissues during implantation. This problem can be especially acute during insertion of a porous implant through a small soft tissue incision, such as those increasingly employed in minimally invasive surgery (“MIS”) procedures.

### SUMMARY OF THE INVENTION

The present invention provides an apparatus including a medical implant. The medical implant includes a porous outer portion, and the apparatus further includes a dried, lubricious when wet, in vivo absorbable coating covering the porous outer portion.

In an alternative embodiment, the present invention provides a method for finishing a medical implant. The method includes forming a porous outer portion on the implant, covering the porous outer portion with a lubricious when wet, in vivo absorbable coating, and drying the coating.

The above-noted features and advantages of the present invention, as well as additional features and advantages, will be readily apparent to those skilled in the art upon reference to the following detailed description and the accompanying drawings.

### DETAILED DESCRIPTION OF EXEMPLARY EMBODIMENT(S)

In an exemplary embodiment, the present invention includes a medical implant having a porous outer portion (i.e., a “porous implant”). The medical implant may be a porous femoral implant or a porous tibial implant of a knee prosthesis, a porous artificial socket or a porous femoral stem of a hip prosthesis, any other porous orthopedic device, or any other porous medical device. To this end, a variety of suitable porous implants are known. For example, U.S. Pat. No. 6,544,472 to Compton et al. (“Compton”), which is incorporated herein by reference, discloses a porous implant that is suitable for incorporation into the present invention. Additionally, U.S. Pat. No. 5,464,440 to Johansson (“Johansson”), which is incorporated herein by reference, discloses a suitable porous implant. It is noted, however, that although Compton and Johansson disclose particular exemplary embodiments (see, e.g., Compton at column 8, lines 1-8; and Johansson at column 1, lines 53-67 through column 2, lines 1-23) various other suitably shaped/sized porous implants having various other suitable pore sizes/configurations (and manners of making them) are well known.

The present invention further includes a dried, lubricious when wet, in vivo absorbable coating that covers the exterior surfaces of the porous portion of the medical implant. U.S. Pat. No. 6,176,849 to Yang et al. (“Yang”), which is incorporated herein by reference, discloses various hydrogels that are suitable as the coating for the present invention. More particularly, practically any of the water-soluble hydrogels disclosed in Yang will suffice (see, e.g., Yang at column 4, lines 36-66; column 7 at lines 57-63; column 8, lines 25-29; column 9, lines 26-30). The rate of in vivo absorption or dissolution of the coating is controlled by selection of the desired solubility of the hydrogel in a known manner. It is noted, however, that the hydrophobic top coat(s) disclosed in Yang are not essential to the present invention and in the preferred embodiment the coating of the present invention consists merely of a water-soluble hydrogel by itself. Nevertheless, in alternative embodiments the coating may include one of the hydrophobic top coats disclosed in Yang. Additionally, it is noted that the particular hydrogels disclosed in Yang are merely exemplary. In alternative embodiments, the coating for the present invention may be composed of any suitable dryable, lubricious when wet, in vivo absorbable coating. Further, it is noted that in alternative embodiments the coating of the present invention may include bone promoting agents, antibiotics, and/or any other suitable bioactive agent(s). To these ends, various suitable dryable, lubricious when wet, in vivo absorbable coatings, bioactive agents, and manners of impregnating such coatings with such agents are known.

The liquid coating is applied to the porous implant by dipping, spraying, or any other suitable manner and then the coating is dried, preferably using heat. In the preferred embodiment, the porous implant is coated by immersing it in a liquid hydrogel such that its porous portion(s) are completely filled with the hydrogel as well as its exterior surface(s) becoming completely covered with a smooth hydrogel coating. However, in alternative embodiments, only the porous portion(s) may be coated. Additionally, in other alternative embodiments the implant may be coated or overlaid such that the pores remain substantially empty or unfilled. In any event, the coating is applied to any suitable height or thickness over the porous outer surface(s) that is sufficient to ensure that a reasonable amount of the coating may be rubbed off or worn off without uncovering the porous outer surface(s) during insertion and maneuvering of the porous implant as discussed further below. Accordingly, in the preferred embodiment the hydrogel is applied to provide a coating layer having a dried, cured thickness of about 1 millimeter. In the preferred embodiment, the hydrogel is crosslinked or cured by exposure to UV light.

To use the present invention, the dried coating is hydrated with blood, any other suitable body fluid, a suitable saline solution, and/or any other suitable hydrating agent. Upon hydration, the coating becomes lubricious. The lubricated porous implant is inserted through an incision and/or maneuvered around a patient's soft tissue(s). Because the porous portion(s) of the implant are covered with the lubricous coating, injuries to the soft tissues by rough or jagged edges are ameliorated. The water-soluble coating dissolves in vivo and is absorbed by surrounding tissues, which opens the pores for bone ingrowth or any other purpose. Depending on the solubility of the coating, pores may become available almost immediately after insertion of the implant or more gradually thereafter. In any event, the implant is fixed in position in a known manner.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

1. An apparatus, comprising:
a medical implant including a porous outer portion; and
a dried, lubricious when wet, in vivo absorbable coating covering the porous outer portion.

2. The apparatus of claim 1, wherein the coating includes a hydrogel.

3. The apparatus of claim 2, wherein the coating consists essentially of a hydrogel.

4. The apparatus of claim 3, wherein the coating is about 1 millimeter thick.

5. The apparatus of claim 1, wherein the coating is about 1 millimeter thick.

6. The apparatus of claim 1, wherein the coating fills the porous outer portion.

7. The apparatus of claim 1, wherein the coating includes a bioactive agent.

8. The apparatus of claim 1, wherein the porous outer portion includes a plurality of sintered metallic particles.

9. A method for finishing a medical implant, the method comprising the steps of:
forming a porous outer portion on the implant;
covering the porous outer portion with a lubricious when wet, in vivo absorbable coating; and
drying the coating.

10. The method of claim 9, wherein the drying step includes heating the coating.

11. The method of claim 9, wherein the covering step includes applying the coating to a thickness of about 1 millimeter.

12. The method of claim 9, further including the step of impregnating the coating with a bioactive agent.
